(19) United States
(12) Patent Application Publication
(10) Pub. No.: US 2002/0131939 A1
(43) Pub. Date: Sep. 19, 2002

(54) Hair protection compositions

(75) Inventors: David Djerassi, New York, NY (US); Alan Martin Marks, East Brunswick, NJ (US)

Correspondence Address: Stephen M. Haracz, Esq., BRYAN CAVE LLP, 245 Park Avenue, New York, NY, 10167-0034, US

(73) Assignee: ROCHE VITAMINS INC.

(21) Appl. No.: 10/042,876

(22) Filed: Jan. 9, 2002

Related U.S. Application Data
(60) Provisional application No. 60/262,055, filed on Jan. 16, 2001.

(51) Int. Cl. A61K 7/42; A61K 7/44; A61K 7/06; A61K 7/00

(52) U.S. Cl. 424/59; 424/60; 424/70.1; 424/400; 424/401

(57) ABSTRACT

A hair care composition is provided. This hair care composition includes (a) about 0.01% (wt) to about 2.5% (wt) of a UV-A filter; (b) about 0.01% (wt) to about 10% (wt) of a UV-B filter; and (c) about 0.01% (wt) to about 2.5% (wt) of phytantriol. Methods of making and using such compositions are also provided.

### FIELD OF THE INVENTION

The present invention relates to hair care compositions for protecting hair from the damaging effects of ultraviolet (UV) radiation, and hot blow-drying. More particularly, the invention relates to a hair care composition containing a UV-A filter, a UV-B filter, and phytantriol. Methods of making and using such compositions are also provided.

### BACKGROUND OF THE INVENTION

Tensile strength is one commonly accepted measure of the relative health of hair. Environmental insults, such as for example, UV radiation from exposure to sunlight and hot air blow drying, may damage hair, which can be determined by measuring the tensile strength hair.

It is known that hair passes through three phases while being extended under increasing force. The first phase, known as the elastic region, is characterized by reversible extension. Hydrogen bonds are thought to be the ones distributed in this region. The second phase, known as the yield region, is characterized by a partially reversible transformation in which covalent and ionic bonds are likely to be disturbed. Leading to the breaking point of a hair is the third region, also known as the post yield region.

It is also known that the breaking point of hair is related to hair diameter, and may not be an indication of overall hair damage. Thus, this parameter is not accurate or sensitive to measure hair damage. Because the yield region is the one most likely to correlate, in general, with covalent and possibly with disulfide bond breakage in hair, and because these are the types of bonds most notably affected by UV radiation, which results in intrinsic structural damage, evaluation of the yield slope may provide a practical and objective measurement of hair damaged by UV exposure.

Hot air blow drying may also damage hair, such as for example, by causing a reduction in tensile strength of the hair. The damage to the hair may be cumulative when the hair is sequentially exposed to UV radiation followed by hot air blow drying as occurs when one is exposed to the sun during the day and then washes, and blow dries the hair with a hot air blow drier in the evening. Such a sequence of events is typical when, for example, one is on vacation at the beach and is exposed to the sun during the day, and then in the evening washes and hot air blow dries the hair.

To combat the damage caused to the hair by UV radiation, UV filters have been incorporated into hair treatment compositions, such as shampoos, conditioners, and gels. See for example Luther, et al. U.S. Pat. No. 6,090,370. Such products, however, do not offer protection against the damaging effects caused by hot air blow drying the hair alone, or after, a period of time in the sun.

Phytantriol has been used in, for example, hair care products, such as shampoos to provide conditioning and stability to the hair. See e.g., Vinski, et al. U.S. Pat. No. 5,776,443. In addition, various compositions have been disclosed in which phytantriol and one or more UV-filters are present. See e.g., Ribier et al. U.S. Pat. Nos. 5,925,364, 5,667,768, and 5,834,103. None of the above-referenced patents disclose that the combination of UV-A and UV-B filters with phytantriol provide protection, or enhanced protection, of the hair from the damaging effects of sequential exposure to UV radiation followed by hot air blow drying. Accordingly, it would be desirable to have a hair composition that is designed to provide protection to the hair from the damaging effects of sequential exposure to UV radiation followed by hot air blow drying.

### SUMMARY OF THE INVENTION

Accordingly, one embodiment of the invention is A hair care composition containing about 0.01% (wt) to about 2.5% (wt) of a UV-A filter; about 0.01% (wt) to about 10% (wt) of a UV-B filter; and about 0.01% (wt) to about 2.5% (wt) of phytantriol.

Another embodiment of the invention is a process for protecting hair from the effects of UV radiation and hot air blow drying. This process includes (a) applying an effective amount of a composition containing about 0.01% (wt) to about 2.5% (wt) of a UV-A filter, about 0.01% (wt) to about 10.0% (wt) of a UV-B filter, and about 0.01% (wt) to about 2.5% (wt) of phytantriol, to hair prior to exposure to UV radiation and hot air blow drying; and (b) exposing the hair sequentially to the UV radiation followed by hot air blow drying, wherein the hair retains at least about 90% of its tensile strength after exposure to UV radiation and hot air blow drying, when compared to hair untreated with the composition.

A further embodiment of the invention is process for producing a shampoo. This process includes (a) mixing together a UV-A filter, a UV-B filter, phytantriol, an amide, and a non-ionic surfactant at about 65° C. to about 70° C. until a clear composition is formed; (b) mixing an anionic surfactant into the clear composition; (c) mixing an aqueous solution comprising a preservative into the composition of step (b) until the mixture is clear; (d) adjusting the pH of the mixture of step (c) to a pH of from 6.0 to 7.2 with a 50%(vol.) citric acid solution; and (e) adjusting the viscosity of the mixture of step (d) with sodium chloride, until a uniform composition is formed having at least a 80% protection value.

Another embodiment of the invention is a process for producing a hair conditioner. This process includes (a) mixing together a UV-A filter, a UV-B filter, phytantriol, polypropylene glycol, cetyl acid, and stearalkonium chloride at about 75° C.; (b) adding water at a temperature of about 65° C. to the mixture of step (a) with mixing and heating until the mixture returns to a temperature of about 75° C.; (c) cooling the mixture formed in step (b) to about 65° C.; (d) adding a preservative to the mixture of step (c); and (e) allowing the mixture of step (d) to cool to room temperature with mixing until a uniform composition is formed having at least a 80% protection value.

Another embodiment of the invention is a method for reducing loss of tensile strength in hair caused by UV radiation and hot air blow drying. This method includes treating a hair with a composition containing about 0.01% (wt) to about 2.5% (wt) of a UV-A filter, about 0.01% (wt) to about 10% (wt) of a UV-B filter; and about 0.01% (wt) to about 2.5% (wt) of phytantriol, wherein the hair when treated with the composition prior to exposure to UV radiation and hot air blow drying retains about 94% of its tensile strength compared to a hair that is not treated with the composition.

### BRIEF DESCRIPTION OF THE DRAWINGS

FIG. 1 is a graph showing the effect on the tensile strength of hair of sequential exposure of the hair to UV radiation followed by hot air blow drying.

FIG. 2 is graph showing the % reduction in tensile strength of hair when exposed to different conditions.

FIG. 3 is a graph showing the effect on the tensile strength of hair treated with a hair shampoo composition according to the present invention prior to sequential exposure to UV radiation followed by hot air blow drying.

FIG. 4 is a graph showing the effect on the tensile strength of hair treated with a hair conditioner composition according to the present invention prior to sequential exposure to UV radiation followed by hot air blow drying.

FIG. 5 is a graph showing the % reduction of hair tensile strength on hair treated with a hair conditioner composition according to the present invention prior to exposure to UV radiation followed by hot air blow drying.

FIG. 6 is a graph showing the % reduction of hair tensile strength on hair treated with a hair shampoo composition according to the present invention prior to sequential exposure to UV radiation followed by hot air blow drying.

FIG. 7 is a graph showing the percent protection (% protection) provided to hair treated with a shampoo according to the present invention prior to sequential exposure to UV radiation followed by hot air blow drying.

### DETAILED DESCRIPTION OF THE INVENTION

The present invention provides a hair care composition that includes a UV-A filter, a UV-B filter, and phytantriol. The composition contains from about 0.1% to about 2.5%(wt) of the UV-A filter, from about 0.01% to about 10%(wt) of the UV-B filter, and from about 0.01% to 2.5%(wt) of phytantriol. Preferably, the composition contains from about 0.1% to about 0.5%(wt) of the UV-A filter, from about 0.1% to about 0.5%(wt) of the UV-B filter, and from about 0.1% to about 0.5%(wt) of phytantriol. More preferably, the composition contains about 0.2%(wt) of the UV-A filter, about 0.5%(wt) of the UV-B filter, and about 0.3%(wt) of phytantriol.

As used herein, the term “UV-A filter” means a compound or composition that absorbs UV radiation in the range of about 320 nm to about 400 nm. Such UV filters include, for example, butyl methoxydibenzoylmethane (PARSOL 1789), 4-(1,1-dimethylethyl)-4′-methoxydibenzoylmethane, 4-isopropyl dibenzoylmethane, 4-methoxy-benzylidene-cyanoacetic acid n-hexyl ester, 4-methoxy-benzylidene-cyanoacetic acid n-octyl ester, 4-methoxy-benzylidene-cyanoacetic acid n-decyl ester, 4-methoxy-benzylidene-cyanoacetic acid isononyl ester, 4-methoxy-benzylidene-cyanoacetic acid isodecyl ester, 4,4′-(6-(bis(2-ethyl-hexyl)-amino)-s-triazine-2,4-diyl)-diresorcinol, 2-(4-Ethoxy-anilinomethylene)-propanedioic acid diethyl ester, Terephtalylidene-3,3′-dicamphor-10,1040 -disulfonic acid, 2-Benzotriazol-2-yl-4-methyl-6-(3-(1,3,3,3-tetramethyl-1-(trimethyl silyl oxy)-disiloxanyl)-2-methyl-propyl)-phenol, 2,2′-(1,4-Phenylen)-bis-1H-benzimidazol-4,6-disulfonic acid, Na salt, Hexyl 2-(4-Diethylamino-2-hydroxy benzoyl) benzoate, Ultrafine ZnO, and mixtures thereof. Preferably, the UV-A filter is butyl methoxydibenzoylmethane (PARSOL 1789).

As used herein, the term “UV-B filter” means a compound or composition that absorbs UV radiation in the range of about 280 nm to about 320 nm. Such UV filters include, for example, octyl methoxycinnamate (PARSOL MCX), 2-ethylhexyl paramethoxycinnamate, oxybenzone, octyl salicylate, p-aminobenzoic acid, p-aminobenzoic acid ethyl ester oxyethylated in ethylene oxide, p-dimethylaminobenzoic acid ethyl ester, p-dimethylaminobenzoic acid amyl ester, p-dimethylaminobenzoic acid 2-ethylhexyl ester, p-dimethylaminobenzoic acid isoamyl ester, p-dimethylaminobenzoic acid hexyl ester, p-dimethylaminobenzoic acid heptyl ester, salicylic acid methyl ester, dipropylene glycol salicylate, salicylic acid homomenthyl ester, salicylic acid 2-ethylhexyl ester, triethanolamine salicylate, N-acetylanthranilic acid trimethylcyclohexyl ester, anthranilic acid menthyl ester, cinnamic acid benzyl ester, cinnamic acid menthyl ester and homomenthyl ester, cinnamic acid octyl ester, p-isopropylcinnamic acid ethyl ester, diisopropylcinnamic acid ethyl ester, diisopropylcinnamic acid methyl ester, p-methoxycinnamic acid isoamyl ester, p-methoxycinnamic acid isopropyl ester, p-methoxycinnamic acid propyl ester, p-methoxycinnamic acid 2-ethylhexyl ester, p-methoxycinnamic acid cyclohexyl ester, p-methoxycinnamic acid and salts, alpha-cyano-beta-phenylcinnamic acid ethyl ester, alpha-cyano-beta-phenylcinnamic acid 2-ethylhexyl ester, 2-hydroxy-4-methoxy-benzophenone, 4-phenylbenzophenone, 4-phenyl-benzophenone-2-carboxylic acid 2-ethylhexyl ester, 2,2′-dihydroxy-4-methoxy-benzophenone, 2-hydroxy-4-n-octyloxybenzophenone, 2,2′-dihydroxy-4,4′-dimethoxy-benzophenone, 2-hydroxy-4-methoxy-benzophenone-5-sulphonic acid, sodium 2,2′-dihydroxy-4,4′-dimethoxy-benzophenone-5-sulphonate, 2,4-dihydroxy-benzophenone, 2,2′-4,4′-tetrahydroxy-benzophenone, 2-hydroxy-4-methoxy-4′-methylbenzophenone, 3-(4-methylbenzylidene)-D,L-camphor, sodium 3-(4-methylbenzylidene)-D,L-camphor-sulphonate, 3-benzylidene-D,L-camphor, m-digallic acid trioleate, 2-phenylbenzimidazole-5-sulphonic acid, sodium 3,4-dimethoxyphenolglyoxylate, beta-imidazole-4(5)-acrylic acid (urocanic acid), 2-phenyl-5-methyl-benzoxazole, dibenzalazine, dianisoylmethyne, 5-(3,3-dimethyl-2-norbornylidene)-3-pent-2-en-2-one, (2′-hydroxy-5′-methylphenyl)-benzotriazole, 1-phenyl-3-(3-pyridyl)-1,3-propanedione, benzylidene malonate silicone polymer (PARASOL SLX), 2,4-Bis((4-(ethyl-hexylox)-2-hydroxy)-phenyl)-6-(4-methoxyphenyl)-1,3,5-triazine (Tinosorb S-2,4-Bis((4-(ethyl-hexylox)-2-hydroxy)-phenyl)-6-(4-methoxyphenyl)-1,3,5-triazine (Tinosorb S-Ciba), 2,2′-Methylene-bis-(6(2H-benzotriazol-2-yl)-4-(1,1,3,3,-tetramethylbutyl)phenol) (Tinosorb M-Ciba), Uvinul T-150 (BASF), UVASORB HEB (3V-Sigma), Ultrafine TiO2, and mixtures thereof. Preferably, the UV-B filter is octyl methoxycinnamate (PARSOL MCX).

Preferably, the hair care composition of the present invention contains butyl methoxydibenzoylmethane (PARSOL 1789), octyl methoxycinnamate (PARSOL MCX), and phytantriol.

The hair care composition may also include a cosmetically acceptable excipient. As used herein, the phrase “cosmetically acceptable excipient” means any compound or composition that is conventionally used to produce other desirable effects in hair care compositions. Thus, in the present invention, cosmetically acceptable excipients include, for example, surfactants, buffering agents to adjust pH, agents to adjust viscosity, emollients, proteins, vitamins, minerals, fragrances, coloring agents, hair bleaches, fillers, and mixtures thereof. For example, in the present invention the cosmetically acceptable excipient may be lauramide DEA, polysorbate 80, sodium laureth sulfate, sodium chloride, citric acid, sodium EDTA, propylene glycol, cetyl alcohol, stearalkonium chloride, methylchloroisothiazolinone, methylisothiazolinone, water, or mixtures thereof.

Thus, the hair care composition containing a UV-A filter, a UV-B filter, and phytantriol may also contain lauramide DEA, polysorbate 80, sodium laureth sulfate, sodium chloride, citric acid, disodium EDTA, methylchloroisothiazolinone, methylisothiazolinone, and water. Alternatively, the hair care composition may include propylene glycol, cetyl alcohol, stearalkonium chloride, methylchloroisothiazolinone, methylisothiazolinone, and water.

In the present invention, the hair care composition may be incorporated into a variety of hair care products. Such hair care products include, for example, shampoos, hair conditioners, hair gels, hair mousses, hair sprays, and hair pomades. Preferably, the hair care composition is incorporated into a hair shampoo or a hair conditioner. As used herein, a “hair shampoo” is any composition that contains at least on surfactant suitable for cleaning hair, such as for example, the shampoo compositions set forth in the examples below.

In the present invention, a “hair conditioner” is any composition that is used to restore the original condition of hair. Such conditioners include, for example, silicones, cationic surfactants and quaternary ammonium compounds, and synthetic cationic polymers. Other components which have been described include moisturizing agents, thickeners or viscosity modifying agents for enhancing hand application, lathering agents for increasing foaming, foam stabilizers, and pearlizing agents. Examples of yet other components which are commonly included include perfumes, pH control agents, colorants, preservatives, and antimicrobials.

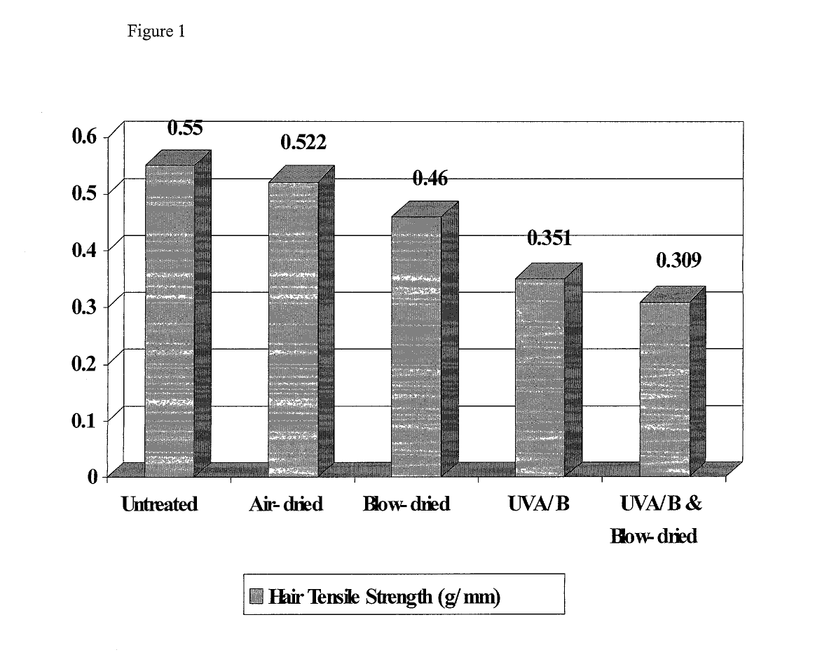

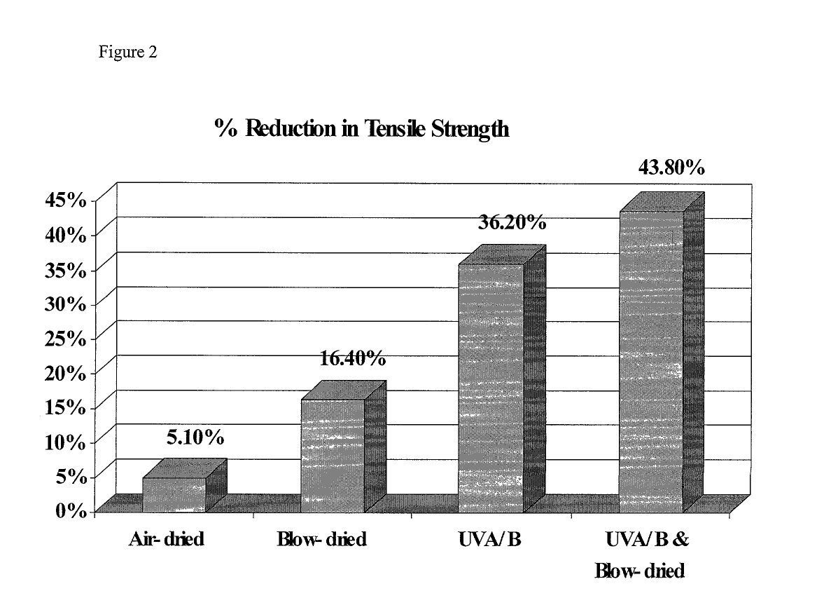

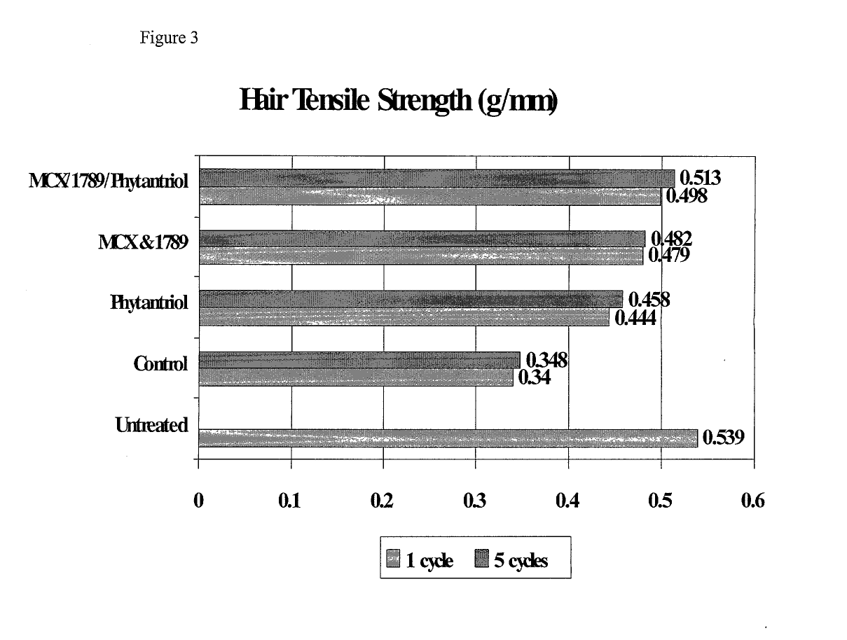

In the present invention, the compositions provide “protection” to the hair. As used herein, “protects,” “protection,” “protected,” and “protecting” are used interchangeably to mean that the compositions disclosed herein reduce the damaging effects of UV radiation and hot air blow drying to the hair. Such damaging effects include reduction in the tensile strength of the hair. For example, FIG. 1 (g/mm) and FIG. 2 (% reduction) show the effect on hair tensile strength of various environmental insults. Thus, the protection afforded to the hair by the compositions of the present invention is measured by the reduction in loss of tensile strength of hair treated with the present compositions prior to sequential exposure to UV radiation followed by hot air blow drying compared to untreated hair samples or samples treated with various other compositions. See e.g., FIG. 3.

In the present invention, hair is “treated” with the compositions disclosed herein. Thus, the term “treated” means that the present compositions are applied to the hair using conventional methods, in sufficient quantities, and for a sufficient amount of time to achieve the desired protection. For example, in the case of a shampoo composition of the present invention, the hair is “treated” by applying an effective amount of the shampoo to the hair e.g., manually or by spraying, etc. The shampoo is then worked into the hair so that substantially all of the hair is contacted with the shampoo. The shampoo is then rinsed out of the hair. As used herein, one application of, e.g., shampoo followed by a rinsing is designated as “one cycle.” In the present invention, at least one cycle is required to treat the hair. Preferably, from 1 to 5 cycles may be used to treat the hair.

Another embodiment of the present invention is a process for protecting hair from the effects of sequential exposure to UV radiation followed by hot air blow drying. This process includes applying an effective amount of a composition containing from about 0.01% to about 2.5%(wt) of a UV-A filter, from about 0.01% to about 10%(wt) of a UV-B filter, and from about 0.01% to 2.5%(wt) of phytantriol to the hair prior to sequential exposure to UV radiation and hot air blow drying.

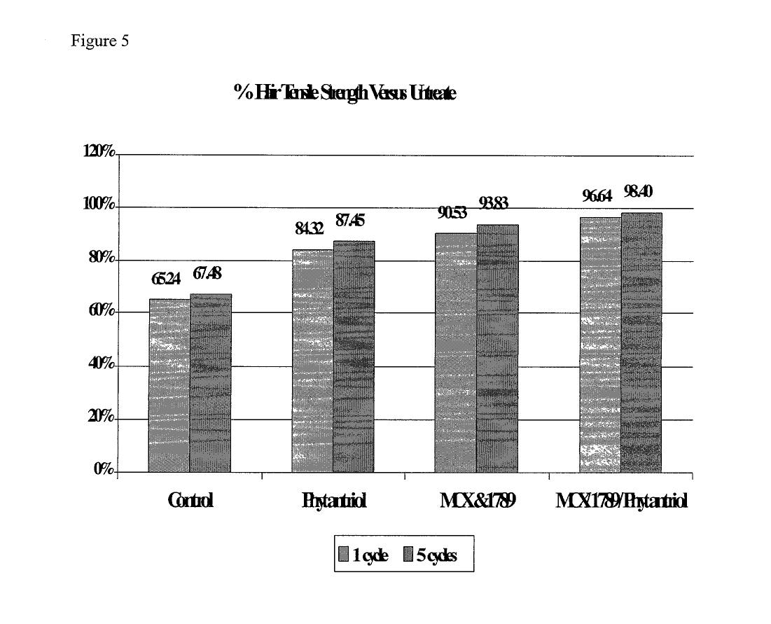

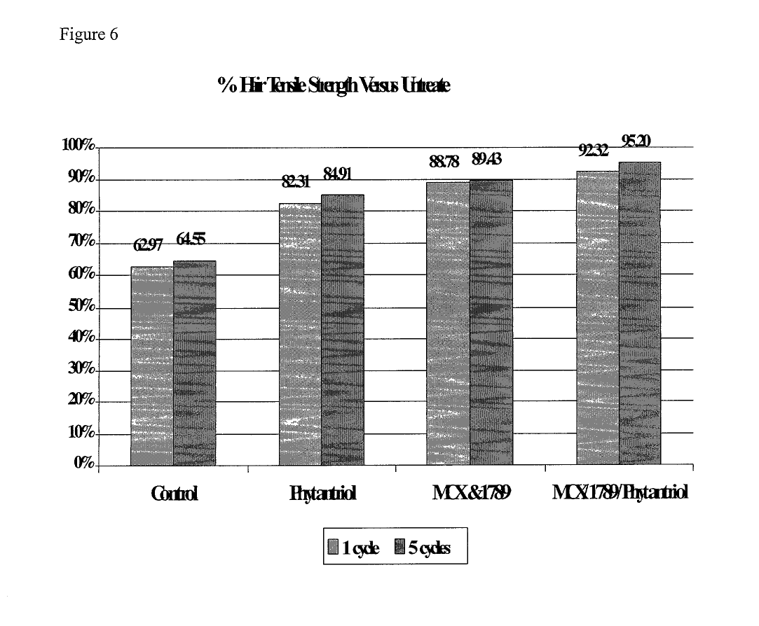

The hair treated with a composition of the present invention is exposed to UV radiation, by for example, spending time in the sun. Thereafter, the hair is further exposed to hot air blow drying. Such a sequence of events is typical of e.g., someone on vacation, who spends the day at the beach, then washes his or her hair, and blows it dry with a hot air blow dryer. In this process, when hair is treated with a composition according to the present invention, the hair retains at least about 95% (conditioner) or at least 90% (shampoo) of its tensile strength after exposure to UV radiation and hot air blow drying, when compared to hair untreated with the composition, and then exposed to UV radiation and hot air blow drying. See FIGS. 5 and 6.

In this process, the composition preferably contains from about 0.1% to about 0.5%(wt) of the UV-A filter, from about 0.1% to about 0.5%(wt) of the UV-B filter, and from about 0.1% to about 0.5%(wt) of phytantriol. More preferably, the composition contains about 0.2%(wt) of the UV-A filter, about 0.5%(wt) of the UV-B filter, and about 0.3%(wt) of phytantriol.

In this process, it is preferred that the UV-A filter is butyl methoxydibenzoylmethane (PARSOL 1789), and that the UV-B filter is octyl methoxycinnamate (PARSOL MCX).

The present invention also provides a process for producing a shampoo. This process includes combining a UV-A filter, a UV-B filter, phytantriol, an amide, and a non-ionic surfactant at about 65° C. to about 70° C. until a clear composition is formed. In this process, the combining step may be achieved by mixing the components in any conventional mixing apparatus. An anionic surfactant is then mixed into the clear composition. Next, an aqueous solution containing a preservative is mixed into the composition until it is clear. The pH of the mixture is then adjusted to between about 6.0 to about 7.2 with, e.g., a 50% (vol.) citric acid solution. The viscosity of the mixture is adjusted with, for example, sodium chloride, and mixed until a uniform composition is formed. Such a composition imparts about 80% protection to the hair from the effects of sequential exposure to UV radiation followed by hot air blow drying.

In the present invention, percent protection (“% protection”) is measured by dividing the tensile strength (in g/mm) of a hair sample that is untreated and not exposed to UV radiation or by hot air blow drying (control sample) by the tensile strength of a hair sample that is treated with a composition according to the present invention prior to sequential exposure to UV radiation followed by hot air blow drying (treated sample). Thus, % protection is provided by the following formula:

(treated sample/control sample)*100%

In the present invention, “% protection values” are assigned to compositions that when used to treat hair provide a certain % protection as defined above. It is preferred that the present compositions have a % protection value of at least 80% or greater, more preferably of at least 88%.

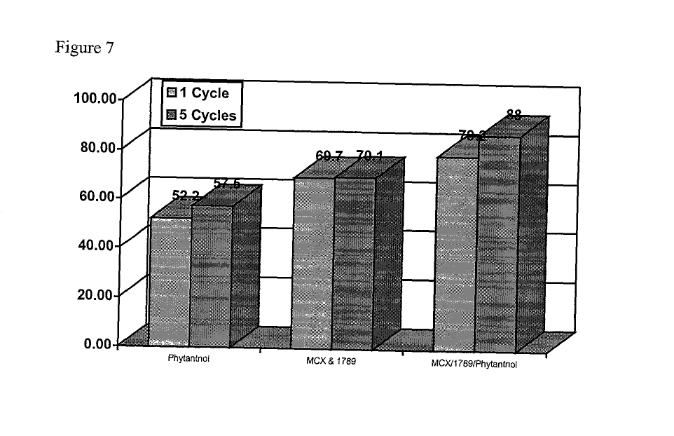

As FIG. 7 demonstrates, a shampoo composition according to the present invention provides 79.2% and 88% protection respectively, from damage caused by UV exposure followed by hot air blow drying. This is a substantial improvement over phytantriol alone or a combination of PARSOL MCX and PARSOL 1789 alone.

In this process, it is preferred that the UV-A filter is butyl methoxydibenzoylmethane (PARSOL 1789), and the UV-B filter is octyl methoxycinnamate (PARSOL MCX). It is also preferred that the amide is lauramide DEA, the non-ionic surfactant is polysorbate 80, and the anionic surfactant is sodium laureth sulfate. The aqueous solution may also contain disodium EDTA, methylchloroisothiazolinone, and methylisothiazolinone.

A hair shampoo may be produced using the process set forth above, wherein the shampoo includes 0.2%(wt) of a UV-A filter; 0.5%(wt) of a UV-B filter; 0.3%(wt) of phytantriol; 30%(wt) sodium laureth sulfate; 3.0%(wt) lauramide DEA; 0.10%(wt) disodium EDTA; 0.25%(wt) of a 50% solution of citric acid; 1.0%(wt) sodium chloride; 3.0%(wt) polysorbate 80; 0.05%(wt) of methylchloroisothiazolinone and methylisothiazolinone; and 61.6%(wt) water.

Another embodiment of the invention is a process for producing a hair conditioner containing a UV-A filter, a UV-B filter, and phytantriol. (FIG. 6). Other appropriate components for a hair conditioner may also be included in the hair conditioner composition. For example, in one aspect of this process, the UV-A filter, the UV-B filter, phytantriol, polypropylene glycol, cetyl acid, and stearalkonium chloride are mixed at a temperature of about 75° C. Water at a temperature of about 65° C. is added with mixing, and the resulting mixture is returned to a temperature of about 75° C. The mixture is cooled to about 65° C. An aqueous solution of a preservative is then added to the mixture. The mixture is allowed to cool to room temperature, with mixing, until a uniform composition is achieved. Such a composition imparts about 80% protection from the effects of sequential exposure to UV radiation followed by hot air blow drying.

Preferably, the UV-A filter is butyl methoxydibenzoylmethane (PARSOL 1789) and the UV-B filter is octyl methoxycinnamate (PARSOL MCX). In this process, the preservative is an aqueous solution containing methylchloroisothiazolinone and methylisothiazolinone. For this process, it is preferred to use the following compounds (in %wt): 0.2% of a UV-A filter; 0.5% of a UV-B filter; 0.3% of phytantriol; 5.0% propylene glycol; 0.5% cetyl alcohol; 1.0% stearalkonium chloride; 0.05% of methylchloroisothiazolinone and methylisothiazolinone; and 90.45% water.

A further embodiment of the invention is a method for reducing the loss of tensile strength in hair caused by sequential exposure to UV radiation followed by hot air blow drying. In this method, hair is first treated with a composition containing (in %wt) from about 0.01% to about 2.5% of the UV-A filter, from about 0.01% to about 10% of the UV-B filter, and from about 0.01% to 2.5% of phytantriol, prior to exposure to UV radiation and hot air blow drying. In this method the hair retains at least 80% of its tensile strength as compared to the control (untreated/unexposed hair).

The following examples are provided to further illustrate the compositions processes and methods of the present invention. These examples are illustrative only and are not intended to limit the scope of the invention in any way.

### EXAMPLES

### Example 1

### UV Radiation and Hot Air Blow Drying

Hair swatches (Virgin Brown hair) of approximately 20 cm in length were used in the following examples.

To simulate one week worth of exposure to UV radiation, hair swatches were placed 26 inches from a Kratos Solar Simulator (______) set at 20 volts and 30 amperes of current for three hours.

Blow drying of the hair swatches using hot air was performed by blowing hot air on the hair at a distance of 12 inches with a hair dryer at a high heat setting for two minutes. This was intended to simulate one weeks worth of blow drying.

In the following examples, the tensile strength of the hair was measured over the yield region. A single strand of hair 20 cm in length was extended with a force at a load setting of 20 g at a speed of 100 mm/minute. For each treatment group or control, the tensile strength of 10 strands of hair were measured and the mean yield region slope was calculated.

### Example 2

### Shampoo Formulation

A shampoo formulation according to the present invention (“Inventive Composition”) was made as set forth in Table 1 below:

TABLE 1

| | | Phytant- | UV-A/B | |
|---|---|---|---|---|
| Ingredients | Inventive Shampoo | riol Only | Only | Control |
| PARSOL MCX | 0.5% | — | 0.5% | — |
| PARSOL 1789 | 0.2% | — | 0.2% | — |
| Phytantriol | 0.3% | 0.3% | — | — |
| Sodium Laureth | 30.0% | 30.0% | 30.0% | 30.0% |
| Sulfate | | | | |
| Monamid 716 | 3.0% | 3.0% | 3.0% | 3.0% |
| Versene Na₂ | 0.1% | 0.1% | 0.1% | 0.1% |
| Citric Acid 50% | 0.25% | 0.25% | 0.25% | 0.25% |
| Sodium Chloride | 1.0% | 1.0% | 1.0% | 1.0% |
| Tween 80 | 3.0% | 3.0% | 3.0% | 3.0% |
| Kathon CG | 0.05% | 0.05% | 0.05% | 0.05% |
| Deionized Water | 61.6% | 62.3% | 61.9% | 62.6% |

Undamaged hair was treated with the respective shampoos set forth in Table 1 for 1 or 5 cycles. In each cycle, hair swatches were soaked in 25% shampoo solution for 2 minutes with constant agitation and then rinsed twice with warm water (40° C.). The treated hair was then sequentially exposed to UV radiation followed by hot air blow drying as set forth in Example 1.

The tensile strength of the hair swatches were measured using the procedures set forth in Example 1, and the measurements are set forth in Table 2 below:

TABLE 2
Hair Tensile Strength (gm/mm)

| | | Hair Pretreated with the Following Shampoos | | | | | | | |
|---|---|---|---|---|---|---|---|---|---|
| Hair | | Inventive Shampoo | | Phytantriol Only | | No Phytantriol or UV Filter | | UV-A/UV-B Only | |
| Sample | Untreated | 1 cycle | 5 cycle | 1 cycle | 5 cycle | 1 cycle | 5 cycle | 1 cycle | 5 cycle |
| 1 | 0.542 | 0.523 | 0.535 | 0.450 | 0.443 | 0.374 | 0.383 | 0.507 | 0.510 |
| 2 | 0.528 | 0.484 | 0.512 | 0.401 | 0.411 | 0.321 | 0.326 | 0.459 | 0.462 |
| 3 | 0.514 | 0.495 | 0.508 | 0.410 | 0.440 | 0.336 | 0.347 | 0.468 | 0.471 |
| 4 | 0.526 | 0.516 | 0.533 | 0.412 | 0.451 | 0.350 | 0.362 | 0.502 | 0.506 |
| 5 | 0.547 | 0.467 | 0.478 | 0.381 | 0.406 | 0.307 | 0.315 | 0.448 | 0.452 |
| 6 | 0.556 | 0.488 | 0.507 | 0.403 | 0.421 | 0.335 | 0.348 | 0.484 | 0.487 |
| 7 | 0.549 | 0.524 | 0.536 | 0.458 | 0.460 | 0.362 | 0.368 | 0.515 | 0.518 |
| 8 | 0.553 | 0.496 | 0.513 | 0.407 | 0.421 | 0.328 | 0.336 | 0.476 | 0.481 |
| 9 | 0.531 | 0.479 | 0.485 | 0.381 | 0.405 | 0.318 | 0.324 | 0.432 | 0.436 |
| 10 | 0.547 | 0.507 | 0.527 | 0.457 | 0.442 | 0.365 | 0.372 | 0.497 | 0.500 |
| Average | 0.539 | 0.498 | 0.513 | 0.416 | 0.430 | 0.340 | 0.348 | 0.479 | 0.482 |
| S.D. | 0.014 | 0.019 | 0.020 | 0.029 | 0.020 | 0.022 | 0.023 | 0.027 | 0.027 |
| t* | | 5.657 | 3.431 | 13.473 | 14.010 | 25.639 | 24.009 | 7.148 | 6.811 |
| p* | | 0.0003 | 0.0075 | <0.0001 | <0.0001 | <0.0001 | <0.0001 | <0.0001 | <0.0001 |

| | | | | | | | | |
|---|---|---|---|---|---|---|---|---|
| % Protection: | 92.32% | 95.20% | 77.14% | 79.73% | 62.97% | 64.55% | 88.78% | 89.43% |

*Compared with untreated hair which has been subjected to hot blow drying.

As Table 2 indicates, the shampoo composition according to the present invention (“Inventive Shampoo) provided significantly better protection (%protection) to the hair compared to controls in both the 1 and 5 cycle experiments. FIG. 3 graphically depicts the enhanced protection conferred by Inventive Shampoo.

### Example 3

### Hair Conditioner Compositions

A hair conditioner composition according to the present invention (“Inventive Conditioner”) and various control compositions were prepared as set forth below:

TABLE 3

| | Inventive | Phytant- | UV-A/B | |
|---|---|---|---|---|
| Ingredients | Conditioner | riol Only | Only | Control |
| PARSOL MCX | 0.5% | — | 0.5% | — |
| PARSOL 1789 | 0.2% | — | 0.2% | — |
| Phytantriol | 0.3% | 0.3% | — | — |
| Propylene Glycol | 5.0% | 5.0% | 5.0% | 5.0% |
| Cetyl Alcohol | 2.5% | 2.5% | 2.5% | 2.5% |
| Stearalkonium | 1.0% | 1.0% | 1.0% | 1.0% |
| Chloride | | | | |
| Kathon CG | 0.05% | 0.05% | 0.05% | 0.05% |
| Deionized Water | 90.45% | 91.15% | 90.75% | 91.45% |

Undamaged hair was treated with the respective hair conditioners for 1 or 5 cycles as set forth in Example 2. In each cycle, hair swatches were soaked in a 50% solution of one of the conditioner formulations set forth in Table 3 above for 2 minutes, and then rinsed twice with warm water (40° C.).

The treated hair was then sequentially exposed to UV radiation and hot air blow drying as set forth in Example 1. The tensile strength of the respective hair swatches were measured using the method set forth in Example 1. The data are presented in Table 4 below:

TABLE 4
Hair Tensile Strength (gm/mm)

| | | Hair Pretreated with the Following Conditioners | | | | | | | |
|---|---|---|---|---|---|---|---|---|---|
| Hair | | Inventive Shampoo | | Phytantriol Only | | No Phytantriol or UV Filter | | UV-A/UV-B Only | |
| Sample | Untreated | 1 cycle | 5 cycle | 1 cycle | 5 cycle | 1 cycle | 5 cycle | 1 cycle | 5 cycle |
| 1 | 0.542 | 0.531 | 0.537 | 0.440 | 0.460 | 0.355 | 0.369 | 0.497 | 0.517 |
| 2 | 0.528 | 0.497 | 0.518 | 0.411 | 0.420 | 0.336 | 0.346 | 0.476 | 0.493 |
| 3 | 0.514 | 0.482 | 0.502 | 0.403 | 0.432 | 0.349 | 0.361 | 0.477 | 0.497 |
| 4 | 0.526 | 0.519 | 0.521 | 0.422 | 0.441 | 0.352 | 0.376 | 0.493 | 0.524 |
| 5 | 0.547 | 0.523 | 0.531 | 0.423 | 0.423 | 0.360 | 0.368 | 0.483 | 0.497 |
| 6 | 0.556 | 0.537 | 0.544 | 0.418 | 0.430 | 0.328 | 0.337 | 0.463 | 0.474 |
| 7 | 0.519 | 0.508 | 0.515 | 0.453 | 0.457 | 0.368 | 0.375 | 0.502 | 0.523 |
| 8 | 0.553 | 0.536 | 0.540 | 0.411 | 0.439 | 0.343 | 0.352 | 0.485 | 0.496 |
| 9 | 0.531 | 0.514 | 0.526 | 0.430 | 0.438 | 0.341 | 0.354 | 0.474 | 0.485 |
| 10 | 0.547 | 0.536 | 0.543 | 0.439 | 0.460 | 0.367 | 0.381 | 0.505 | 0.526 |
| Average | 0.536 | 0.518 | 0.528 | 0.425 | 0.440 | 0.350 | 0.362 | 0.486 | 0.503 |
| S.D. | 0.015 | 0.018 | 0.014 | 0.016 | 0.015 | 0.013 | 0.014 | 0.014 | 0.018 |
| t* | | 6.587 | 6.074 | 16.375 | 14.610 | 27.162 | 23.801 | 7.571 | 3.977 |
| p* | | 0.0001 | 0.0002 | <0.0001 | <0.0001 | <0.0001 | <0.0001 | <0.0001 | 0.0032 |

| | | | | | | | | |
|---|---|---|---|---|---|---|---|---|
| % Protection: | 96.64% | 98.40% | 79.25% | 82.04% | 65.24% | 67.48% | 90.53% | 93.83% |

*Compared with untreated hair which has been subjected to hot blow drying.

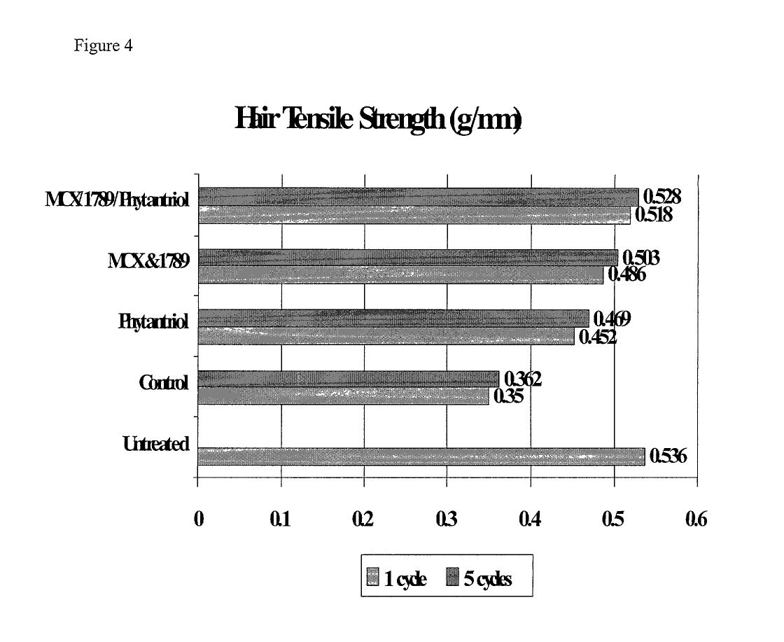

As Table 4 indicates, the Inventive Conditioner provided significantly better protection (%protection) to the hair compared to controls in both the 1 and 5 cycle experiments. FIGS. 4 and 5 graphically depict the enhanced protection conferred by Inventive Conditioner according to the present invention.

The invention being thus described, it will be obvious that the same may be varied in many ways. Such variations are not to be regarded as a departure from the spirit and scope of the invention and all such modifications are intended to be included within the scope of the following claims.

What is claimed is:

1. A hair care composition comprising:
(a) about 0.01% (wt) to about 2.5% (wt) of a UV-A filter;
(b) about 0.01% (wt) to about 10% (wt) of a UV-B filter; and
(c) about 0.01% (wt) to about 2.5% (wt) of phytantriol.

2. A composition according to claim 1 wherein:
(a) the amount of UV-A filter in the composition is about 0.1% (wt) to about 0.5% (wt);
(b) the amount of UV-B filter in the composition is about 0.1% (wt) to about 0.5% (wt); and
(c) the amount of phytantriol in the composition is about 0.1% (wt) to about 0.5% (wt).

3. A composition according to claim 1 wherein:
(a) the amount of UV-A filter in the composition is about 0.2% (wt);
(b) the amount of UV-B filter in the composition is about 0.5% (wt); and
(c) the amount of phytantriol in the composition is about 0.3% (wt).

4. A composition according to claim I wherein the UV-A filter is selected from the group consisting of butyl methoxydibenzoylmethane (PARSOL 1789), 4-(1,1-dimethylethyl)-4′-methoxydibenzoylmethane, 4-isopropyl dibenzoylmethane, 4-methoxy-benzylidene-cyanoacetic acid n-hexyl ester, 4-methoxy-benzylidene-cyanoacetic acid n-octyl ester, 4-methoxy-benzylidene-cyanoacetic acid n-decyl ester, 4-methoxy-benzylidene-cyanoacetic acid isononyl ester, 4-methoxy-benzylidene-cyanoacetic acid isodecyl ester, 4,4′-(6-(bis(2-ethyl-hexyl)-amino)-s-triazine-2,4-diyl)-diresorcinol, 2-(4-Ethoxy-anilinomethylene)-propanedioic acid diethyl ester, Terephtalylidene-3,3′-dicamphor-10,10′-disulfonic acid, 2-Benzotriazol-2-yl-4-methyl-6-(3-(1,3,3,3-tetramethyl-1-(trimethyl silyl oxy)-disiloxanyl)-2-methyl-propyl)-phenol, 2,2′-(1,4-Phenylen)-bis-1H-benzimidazol-4,6-disulfonic acid, Na salt, Hexyl 2-(4-Diethylamino-2-hydroxy benzoyl) benzoate, Ultrafine ZnO, and mixtures thereof.

5. A composition according to claim 4 wherein the UV-A filter is selected from the group consisting of butyl methoxydibenzoylmethane (PARSOL 1789),4,4′-(6)-(bis(2-ethyl-hexyl)-amino)-s-triazine-2,4-diyl)-diresorcinol, 2-(4-Ethoxy-anilinomethylene)-propanedioic acid diethyl ester, and mixtures thereof.

6. A composition according to claim 4 wherein the UV-B filter is selected from the group consisting of octyl methoxycinnamate (PARSOL MCX), 2-ethylhexyl paramethoxycinnamate, oxybenzone, octyl salicylate, p-aminobenzoic acid, p-aminobenzoic acid ethyl ester oxyethylated in ethylene oxide, p-dimethylaminobenzoic acid ethyl ester, p-dimethylaminobenzoic acid amyl ester, p-dimethylaminobenzoic acid 2-ethylhexyl ester, p-dimethylaminobenzoic acid isoamyl ester, p-dimethylaminobenzoic acid hexyl ester, p-dimethylaminobenzoic acid heptyl ester, salicylic acid methyl ester, dipropylene glycol salicylate, salicylic acid homomenthyl ester, salicylic acid 2-ethylhexyl ester, triethanolamine salicylate, N-acetylanthranilic acid trimethylcyclohexyl ester, anthranilic acid menthyl ester, cinnamic acid benzyl ester, cinnamic acid menthyl ester and homomenthyl ester, cinnamic acid octyl ester, p-isopropylcinnamic acid ethyl ester, diisopropylcinnamic acid ethyl ester, diisopropylcinnamic acid methyl ester, p-methoxycinnamic acid isoamyl ester, p-methoxycinnamic acid isopropyl ester, p-methoxycinnamic acid propyl ester, p-methoxycinnamic acid 2-ethylhexyl ester, p-methoxycinnamic acid cyclohexyl ester, p-methoxycinnamic acid and salts, alpha-cyano-beta-phenylcinnamic acid ethyl ester, alpha-cyano-beta-phenylcinnamic acid 2-ethylhexyl ester, 2-hydroxy-4-methoxy-benzophenone, 4-phenylbenzophenone, 4-phenyl-benzophenone-2-carboxylic acid 2-ethylhexyl ester, 2,2′-dihydroxy-4-methoxy-benzophenone, 2-hydroxy-4-n-octyloxybenzophenone, 2,2′-dihydroxy-4,4′-dimethoxy-benzophenone, 2-hydroxy-4-methoxy-benzophenone-5-sulphonic acid, sodium 2,2′-dihydroxy-4,4′-dimethoxy-benzophenone-5-sulphonate, 2,4-dihydroxy-benzophenone, 2,2′-4,4′-tetrahydroxy-benzophenone, 2-hydroxy-4-methoxy-4′-methylbenzophenone, 3-(4-methylbenzylidene)-D,L-camphor, sodium 3-(4-methylbenzylidene)-D,L-camphor-sulphonate, 3-benzylidene-D,L-camphor, m-digallic acid trioleate, 2-phenylbenzimidazole-5-sulphonic acid, sodium 3,4-dimethoxyphenolglyoxylate, beta-imidazole-4(5)-acrylic acid (urocanic acid), 2-phenyl-5-methyl-benzoxazole, dibenzalazine, dianisoylmethyne, 5-(3,3-dimethyl-2-norbornylidene)-3-pent-2-en-2-one, (2′-hydroxy-5′-methylphenyl)-benzotriazole, 1-phenyl-3-(3-pyridyl)-1,3-propanedione, benzylidene malonate silicone polymer (PARASOL SLX), 2,4-Bis((4-(ethyl-hexylox)-2-hydroxy)-phenyl)-6-(4-methoxyphenyl)-1,3,5-triazine (Tinosorb S), 2,2′-Methylene-bis-(6(2H-benzotriazol-2-yl)-4-(1,1,3,3,-tetramethylbutyl)phenol) (Tinosorb M), Uvinul T-150, UVASORB HEB, Ultrafine TiO₂, and mixtures thereof.

7. A composition according to claim 6 wherein the UV-B filter is octyl methoxycinnamate (PARSOL MCX) or benzylidene malonate silicone polymer (PARASOL SLX).

8. A composition according to claim 1 wherein the UV-A filter is selected from the group consisting of butyl methoxydibenzoylmethane (PARSOL 1789), 4,4′-(6-(bis(2-ethyl-hexyl)-amino)-s-triazine-2,4-diyl)-diresorcinol (Triazin), 2-(4-Ethoxy-anilinomethylene)-propanedioic acid diethyl ester, and mixtures thereof, and the UV-B filter is octyl methoxycinnamate (PARSOL MCX) or benzylidene malonate silicone polymer (PARASOL SLX).

9. A composition according to claim 8 wherein the UV-A filter is butyl methoxydibenzoylmethane (PARSOL 1789) and the UV-B filter is octyl methoxycinnamate (PARSOL MCX).

10. A composition according to claim 1 further comprising a cosmetically acceptable excipient.

11. A composition according to claim 10 wherein the cosmetically acceptable excipient is selected from the group consisting of lauramide DEA, polysorbate 80, sodium laureth sulfate, sodium chloride, citric acid, disodium EDTA, propylene glycol, cetyl alcohol, stearalkonium chloride, methylchloroisothiazolinone, methylisothiazolinone, water, and mixtures thereof.

12. A composition according to claim 1 further comprising lauramide DEA, polysorbate 80, sodium laureth sulfate, sodium chloride, citric acid, disodium EDTA, methylchloroisothiazolinone, methylisothiazolinone, and water.

13. A composition according to claim 1 further comprising propylene glycol, cetyl alcohol, stearalkonium chloride, methylchloroisothiazolinone, methylisothiazolinone, and water.

14. A composition according to claim 1 which is incorporated into a hair care product selected from the group consisting of a shampoo, a conditioner, a gel, a mousse, a hair spray, and a hair pomade.

15. A composition according to claim 14 wherein the composition is incorporated into a hair shampoo or a hair conditioner.

16. A process for protecting hair from the effects of UV radiation and hot air blow drying comprising:
(a) applying an effective amount of a composition comprising about 0.01% (wt) to about 2.5% (wt) of a UV-A filter, about 0.01% (wt) to about 10.0% (wt) of a UV-B filter, and about 0.01% (wt) to about 2.5% (wt) of phytantriol, to hair prior to exposure to UV radiation and hot air blow drying; and
(b) exposing the hair sequentially to the UV radiation followed by hot air blow drying, wherein the hair retains at least about 90% of its tensile strength after exposure to UV radiation and hot air blow drying, when compared to hair untreated with the composition.

17. A process according to claim 16 wherein:
(a) the amount of UV-A filter in the composition is about 0.1% (wt) to about 0.5% (wt);
(b) the amount of UV-B filter in the composition is about 0.1% (wt) to about 0.5% (wt); and
(c) the amount of phytantriol in the composition is about 0.1% (wt) to about 0.5% (wt).

18. A process according to claim 16 wherein:
(a) the amount of UV-A filter in the composition is about 0.2% (wt);
(b) the amount of UV-B filter in the composition is about 0.5% (wt); and
(c) the amount of phytantriol in the composition is about 0.3% (wt).

19. A process according to claim 16 wherein the UV-A filter is butyl methoxydibenzoylmethane (PARSOL 1789), 4,4′-(6-(bis(2-ethyl-hexyl)-amino)-s-triazine-2,4-diyl)-diresorcinol (Triazin), 2-(4-Ethoxy-anilinomethylene)-propanedioic acid diethyl ester, and mixtures thereof, and the UV-B filter is octyl methoxycinnamate (PARSOL MCX) or benzylidene malonate silicone polymer (PARASOL SLX).

20. A process according to claim 19 wherein the UV-A filter is butyl methoxydibenzoylmethane (PARSOL 1789) and the UV-B filter is octyl methoxycinnamate (PARSOL MCX).

21. A process for producing a shampoo comprising:
(a) mixing together a UV-A filter, a UV-B filter, phytantriol, an amide, and a non-ionic surfactant at about 65° C. to about 70° C. until a clear composition is formed;
(b) mixing an anionic surfactant into the clear composition;
(c) mixing an aqueous solution comprising a preservative into the composition of step (b) until the mixture is clear;
(d) adjusting the pH of the mixture of step (c) to a pH of from 6.0 to 7.2 with a 50%(vol.) citric acid solution; and
(e) adjusting the viscosity of the mixture of step (d) with sodium chloride, until a uniform composition is formed having at least a 80% protection value.

22. A process according to claim 21 wherein the UV-A filter is butyl methoxydibenzoylmethane (PARSOL 1789), 4,4′-(6-(bis(2-ethyl-hexyl)-amino)-s-triazine-2,4-diyl)-diresorcinol (Triazin), 2-(4-Ethoxy-anilinomethylene)-propanedioic acid diethyl ester, and mixtures thereof, and the UV-B filter is octyl methoxycinnamate (PARSOL MCX) or benzylidene malonate silicone polymer (PARASOL SLX).

23. A process according to claim 22 wherein the UV-A filter is butyl methoxydibenzoylmethane (PARSOL 1789) and the UV-B filter is octyl methoxycinnamate (PARSOL MCX).

24. A process according to claim 21 wherein the amide is lauramide DEA, the non-ionic surfactant is polysorbate 80, and the anionic surfactant is sodium laureth sulfate.

25. A process according to claim 21 wherein the aqueous solution in step (c) comprises disodium EDTA, methylchloroisothiazolinone, and methylisothiazolinone in water.

26. A hair shampoo formed by the process of claim 21 comprising by weight:
(a) 0.2% of a UV-A filter;
(b) 0.5% of a UV-B filter;
(c) 0.3% of phytantriol;
(d) 30% sodium laureth sulfate;
(e) 3.0% lauramide DEA;
(f) 0.10% disodium EDTA;
(g) 0.25% of a 50% (wt) solution of citric acid;
(h) 1.0% sodium chloride;
(i) 3.0% polysorbate 80;
(j) 0.05% of methylchloroisothiazolinone and methylisothiazolinone; and
(k) 61.6% water.

27. A process for producing a hair conditioner comprising:
(a) mixing together a UV-A filter, a UV-B filter, phytantriol, polypropylene glycol, cetyl acid, and stearalkonium chloride at about 75° C.;
(b) adding water at a temperature of about 65° C. to the mixture of step (a) with mixing and heating until the mixture returns to a temperature of about 75° C.;
(c) cooling the mixture formed in step (b) to about 65° C.;
(d) adding a preservative to the mixture of step (c); and
(e) allowing the mixture of step (d) to cool to room temperature with mixing until a uniform composition is formed having at least a 80% protection value.

28. A process according to claim 27 wherein the UV-A filter is butyl methoxydibenzoylmethane (PARSOL 1789), 4,4′-(6-(bis(2-ethyl-hexyl)-amino)-s-triazine-2,4-diyl)-diresorcinol (Triazin), 2-(4-Ethoxy-anilinomethylene)-propanedioic acid diethyl ester, and mixtures thereof, and the UV-B filter is octyl methoxycinnamate (PARSOL MCX) or benzylidene malonate silicone polymer (PARASOL SLX).

29. A process according to claim 28 wherein the UV-A filter is butyl methoxydibenzoylmethane (PARSOL 1789) and the UV-B filter is octyl methoxycinnamate (PARSOL MCX).

30. A process according to claim 27 wherein the preservative is a solution containing methylchloroisothiazolinone and methylisothiazolinone.

31. A hair conditioner formed by the process of claim 27 comprising by weight:
(a) 0.2% of a UV-A filter;
(b) 0.5% of a UV-B filter;
(c) 0.3% of phytantriol;
(d) 5.0% propylene glycol;
(e) 2.5% cetyl alcohol;
(f) 1.0% stearalkonium chloride;
(g) 0.05% of methylchloroisothiazolinone and methylisothiazolinone; and
(h) 90.45% water.

32. A method for reducing loss of tensile strength in hair caused by UV radiation and hot air blow drying comprising treating a hair with a composition comprising about 0.01% (wt) to about 2.5% (wt) of a UV-A filter, about 0.01% (wt) to about 10% (wt) of a UV-B filter; and about 0.01% (wt) to about 2.5% (wt) of phytantriol, wherein the hair when treated with the composition prior to exposure to UV radiation and hot air blow drying retains about 94% of its tensile strength compared to a hair that is not treated with the composition.

33. A method according to claim 32 wherein the treating step comprises applying the composition to the hair from one to five times.

34. A method according to claim 32 wherein the treating step comprises applying the composition to the hair to achieve a % protection value of at least 80%.
